(19) United States
(12) Patent Application Publication
(10) Pub. No.: US 2002/0099440 A1
(43) Pub. Date: Jul. 25, 2002

(54) Individual vein valve prosthesis

(76) Inventors: Augustinus Bader, Immensen (DE); Lars Sunnanvader, Hechingen (DE)

Correspondence Address: CLARK & ELBING LLP, 101 FEDERAL STREET, BOSTON, MA, 02110, US

(21) Appl. No.: 09/905,621

(22) Filed: Jul. 13, 2001

(30) Foreign Application Priority Data
Jul. 14, 2000 (DE) 10034583.2-45

(51) Int. Cl. A61F 2/06

(52) U.S. Cl. 623/1.41; 623/1.24

(57) ABSTRACT

The invention relates to the use of a natural tissue matrix which has undergone recipient-specific transformation for producing an individual vein valve prosthesis without the natural tissue matrix having been acellularized.

The invention relates to the use of a natural tissue matrix which has undergone recipient-specific transformation for producing an individual vein valve prosthesis without the natural tissue matrix having been acellularized.

The replacement of valve systems in the body represents a serious clinical problem especially in the small flow regions, in particular in venous regions with low volumetric flow, because of the risk of thrombosis and long times for incorporation of the implants. Attempts have been made in the past, without success, to produce vein valves which have been treated with tannins and fixatives (e.g. glutaraldehyde) in correspondence with the heart valves which can be employed in the large vessel regions. However, it was noticed that this methods leads, because of the great thrombogenicity of the surfaces, to a high risk of occlusion after implantation in the small flow regions. This is evident and provoked in particular also by the lack of integration at the suture sites. This has led to considerations involving a lengthening of the implant. This means that the vein valves and their attachments above and below the valve ring must be, viewed proportionally, considerably longer than was already possible for heart valves.

Thus, developments which required attachments of more than one times the diameter of the veins, or were in fact even several times longer, were advanced. However, in principle, the clinical situation would require a quite different approach. It is known for therapeutic implants that only as much foreign material as is necessary should be introduced into the body because, in the final analysis, it does mean a foreign body with consequent immunological or regenerative problems and the sequalae associated therewith. They include the thrombogenicity and calcification of the implant. In a second phase it was then attempted also to apply to vein valves developments which indeed show an improved A suitability for larger vessels and heart valves. These include methods and approaches as disclosed in DE 198 28 726 A1. These comprise methods for acellularizing an allogeneic or xenogeneic matrix using enzymatic or else other methods through the use or in conjunction with rinsing processes in the case of vessels. This application already described vessels, with veins also being covered by the definition of vessels, as is generally known. It is likewise an integral part of the state of anatomical knowledge that all veins have vein valves since these are a physiological constituent of the function of veins. The intention thereof is to make directed flow back to the heart possible even against a static load. This has been known for a long time and even very early standard textbooks of anatomy referred thereto.

Although application of the methods disclosed in WO 99/00152 and in DE 199 10 340 A1 to small veins is possible in principle, it leads to the same problems as with heart valves, with the difference that they become of increasing clinical relevance because of the smallness and fragility of the veins in view of the small wall thicknesses, and make use prohibitive. The following disadvantages thereof are of special clinical relevance. On the one hand, the severe destabilization of the matrix through loosening of the fiber bundles and removal of the cement molecules such as, inter alia, fibronectin and laminin between the still remaining main collagen and elastin fibers and through the enzymatic processes leads to a considerable impairment of the suturability of these materials at the time of implantation in the patient. This frequently (⅖cases) leads to tears and leaks after the implantation. Particularly in relation to the thin and thus especially vulnerable walls of the veins of a leg in the region of clinical application, this means that considerable risks of thrombosis remain in such cases. On the other hand, the patent applications DE 198 28 726 A1 and WO 99/00152 disclose that it would be sufficient to take normal endothelial cells and myofibroblasts from a vein of the subsequent recipient and to perform therewith a recolonization with these autologous cells outside the body in order to prepare the subsequent vessel implant optimally for integration into the recipient organism. Normal vessels such as, for example, veins from other regions are available for taking these donor vessels. However, no-one would take cells from an inflamed, i.e. diseased, vein in order to use them to set up a cell culture in vitro in order to produce another new vein because the inflammation is caused, for example, by bacterial processes which would make a culture impossible. Nevertheless, in the conventional procedure disclosed in DE 198 28 726 A1 and in the combinations, described therein, of the various cell types of endothelial cells with myofibroblasts at various sites there are the serious disadvantages that, on the one hand, cells would, according to the current teaching, be taken from a healthy vessel and then would be applied to a matrix which has been acellularized on the other hand. This means that cells with a dormant, i.e. “normal”, phenotype, which does not make them capable of remodeling processes, are taken and then placed in a micro environment which in turn induces a very static state of these cells. The acellularization removes the main initiators of remodeling processes, namely the foreign cells and the cell residues, which generally activate, irrespective of their origin, the local cells via cascade processes in order to be able to change from a dormant into an activated phenotype. This means that the conventional teaching is disadvantageous for the necessary remodeling processes. These local factors of initiation of cascade processes, which require multiple and accurate mediator concentration, interacting temporally and in terms of concentration, by, for example, chemotactic factors, cannot be successfully stimulated even by external additions. The reason for this is that these factors are in part unknown or that the kinetics cannot be complied with. In addition, such factors are costly and would involve long pharmaceutical approval procedures.

WO 95/24873 also discloses methods intended to remove immunologically potentially active cell components. These components with immunogenic activity are, for example, proteins bound to cell membranes. Likewise, EP 0 564 786 A2 deals with various types of acellularization and describes the solution used herein, as well as methods for freezing, storing and rehydrating.

WO 00/32249 follows up the idea that, for recolonization of prostheses, e.g. heart valves, there should be use of attraction factors which are intended to make it possible for endothelial cells to adhere to the surfaces. It thus relates to a prosthesis including a substrate with an associated attraction compound. The latter is intended to attract viable fibroblast precursor cells in vivo, for example also monocytes, and to make possible differentiation to fibroblasts on site and to bind the substrate. In particular, these macrophages are to have specific markers (HLA-DR) so that it is possible selectively to recruit the correct precursor cells for the fibroblast genes. The attraction factors are natural ligands or parts thereof, such as, for example, CD4, HSP-70 (heat shock protein) and TCR (T-cell receptor). Likewise, methods for attracting specific endothelial precursor cells (CD34,Flk-1, endoglin, e-selectin, CD31) are described. According to the disclosure, a specific ligand is always used to attract specific cell populations in the sense of precursor cells. These ligands are bound to the matrix before it is implanted. However, this leads to a considerable reduction of the process and of the problem to specific ligands. The ligands are not at all able to avoid the immunological stimulation processes through allogeneic or xenogeneic cell detritus and, on the contrary, in fact enhance them because implantation is followed by invasion of the cells attracted by these ligands, such as, for example, macrophages. This should be completely avoided in order to avoid an immunological initiated destruction of the implant. In practice, these are by no means desirable, and a method which could completely dispense with these ligand processes would represent a great relief also in relation to approval.

DE 199 10340 A1 describes a method for sheathing veins in the coronary vessel region, which provides a strengthening of the vein wall of a freshly removed vital vein of the recipient with his own cells (fibroblasts, myocytes, endothelial cells) in order to increase the mechanical load-bearing capacity of the vein. This is concerned with another indication region on the heart and therefore not with an avital vein which deliberately contains cell detritus; nor is it intended to induce remodeling. On the contrary, in order to avoid death of the autologous cells already present in the transplant, they are transplanted immediately.

It was therefore an object of the present invent-on essentially to avoid the disadvantages described above.

The present invention therefore relates to the use of a natural tissue matrix which has undergone recipient-specific transformation for producing an individual vein valve prosthesis, without the natural tissue matrix having been acellularized.

Thus, according to the invention, a method which is entirely different from and contrary to the conventional teaching is employed. For this purpose, a native matrix is recolonized in vitro with the cell types capable of interacting, and thus stimulated to this specific interaction, with avoidance of acellularization processes and with avoidance of attraction ligand approaches.

These preferably recipient-compatible and, in particular, autologous cells comprise as basis, in particular fibroblasts, myocytes and endothelial cells. Myofibroblasts are generally initiated even by cell detritus and carry out an activation switch. This is additionally enhanced in particular by the presence of macrophages which have been activated by cell residues or particles and which thus release activation cocktails specific for remodeling processes. These must be distinguished from immunological activation cocktails which would be released in vivo if the foreign cells of allogeneic or xenogeneic origin were to be introduced into an organism in vivo. However, according to the invention, a selective, ie. particle-induced, activation process takes place outside the body in vitro, for example in bioreactors. It is advantageously impossible for leukocytes and plasma cell activations to take place therein.

Since the activation processes generally have two components, on the one hand through the nature and composition of the matrix and through the nature and presence of the activating cells, according to the invention the activation processes can be controlled essentially differently and voluntarily.

Endogenous particle induction by cell residues (detritus) initially still present in the matrix of the subsequent implant can thus be achieved, partly or supplementarily exogenously, by addition of particles. These are preferably smaller than about 15 μm and are added to macrophage cultures, the supernatants of which are then used in order to be able to activate the myofibroblasts on the matrix.

A “recipient-specific transformation” means in this connection preferably a colonization of the selected matrix with recipient-compatible cells, in particular with autologous cells of the prosthesis recipient, and in particular on the basis of the methods of the invention described above with integration of the activation processes.

The matrix which is described hereinafter in detail is colonized with recipient-compatible cells to such an extent that the thrombogenicity of the foreign body “vein valve prosthesis” is sufficiently suppressed. The nature of the colonized cells may also have an effect on the thrombogenicity. Colonization with fibroblasts and endothelial cells alone and, where appropriate, with myofibroblasts too is particularly suitable.

The matrix used for the recipient-specific transformation may also be a matrix which is xenogeneic or allogeneic for the recipient. In this case it is possible to combine exogenous activation processes by phagocytic macrophages.

It is possible and preferred for the basic matrix used for the recipient-specific transformation to be a natural vein valve or artery valve.

It is particularly advantageous for the matrix, in particular the surface of the matrix, to contain microparticles which preferably contain phagocytosable or degradable materials. For example, the surface of the matrix can be completely coated, or the proportion of microparticles to the matrix is about 15-60% or about 15-85%. Examples of suitable materials are polylactides, polyesters, polyurethanes, polyhydroxy-alkanoates, starch or sugar derivatives, lipids, proteins or collagens, preferably polylactides. The diameter of the particles is preferably up to about 15 micrometers (μm). Particularly suitable micro-particles are those having a prolonged half-life of, for example, 2 weeks. Microparticles of this type, for example polylactide particles, are generally obtainable.

In an alternative procedure it is possible for a matrix material which has undergone recipient-specific transformation to be used for constructing a vein valve prosthesis. The procedure for this may be such that the vein valve prosthesis is composed of matrix materials. The selected materials are precolonized before the construction of the vein valve prosthesis and may where appropriate after the construction be coated on the surface in a further step of the method with a material which may also be thrombogenic, such as, for example, extracellular matrix (collagens, fibrin), and additionally colonized further with recipient-specific cells.

The vein valve prosthesis moreover preferably comprises at least one valve leaflet.

In order to ensure better connection to the recipient's vein, the vein valve prosthesis can, in a preferred embodiment, comprise a piece of vein of a certain total length, it preferably being provided for the vein valve to be located in a piece of vein whose length above and below the valve region may also in an advantageous manner correspond to less than the diameter of the vein.

A great advantage of the invention is that the vein valve which has been processed in the stated manner and, where appropriate, newly constructed especially for the recipient can be prepared in steps of the method such that assimilation with the recipient is essentially complete due to the enhanced remodeling which starts even in vitro. This also includes the integration and optimal generation of a continuity of the suture sites at the places which otherwise signify the maximum risk of thrombosis. It is necessary at these places to preclude even slight risks because minimal thromboses would lead to immediate failure of the implant and would cause long-term damage such as venous insufficiency, nutritional disorders and disturbances of wound healing.

It is also advantageous that the function of the vein valve prosthesis can be tested at least in vitro by the vein valve being clamped in an appropriate apparatus and perfused in pulses with a culture medium or a simple (crystalloid) solution.

### EXEMPLARY EMBODIMENTS

### Example 1

After removal of an allogeneic vein it was introduced into a bioreactor apparatus and fixed there. Cells which were autologous in relation to the subsequent implant recipient were expanded in culture bottles observing the usual sterile conditions and standard media for 2 weeks. After detachment from the culture surfaces used for the expansion, these cells were applied luminally in the case of endothelial cells and on the periphery in the case of myofibroblasts of an A allogeneic vein. Standard culture media were used for this purpose. These cells were left there to grow further and integrate in depth for about 1-2 weeks. During this period, the vein was continuously perfused with culture medium, which was renewed every 2 days. It is possible to use cryopreserved or additionally sterilized tissue for the colonization process.

If the vein valve transformation process is to be further expedited it is possible for the entire macrophage population obtained from about 10 ml of a patient's blood by standard isolation methods to be applied to the insides of the bioreactors, i.e. spatially separate from the future implant. These adhere there directly also on plastic surfaces, membranes or glass. The particle and cell wall constituents, which are released during the perfusion of the vein, of the original allogeneic cells bring about activation of macrophages. These secrete cytokines and growth factors directly into the culture medium circulating in the bioreactors. The cytokines released on stimulation are released on site in macrophage-specific kinetics and quantities. This factor cocktail, which is not to be influenced further, in turn has chemotactic and migration-inducing effects on smooth muscle cells and fibroblasts, which can be applied to the periphery of the vein and luminally. This results in directed migration and remodeling processes because the myofibroblasts carry out a phenotypical switch into the so-called secretory state. Destruction processes by macrophages are avoided because they cannot make physical contact with an implant.

### Example 2

A natural tissue matrix which also contains in the surfaces of the tissue fibers a proportion of, for example, 15% of polylactide particles with a diameter of, for example, about 14 μm with a prolonged half-life, and which is preferably maintained for about two weeks longer than the polylactide surface structures which dissolve in the short term, is colonized on both sides with myofibroblasts using conventional nutrient media. The perfusion processes in the bioreactors and the action of the aqueous solutions result in particles being detached from the surface structures without impairing the overall stability. These particles are phagocytosed by the myofibroblasts and transported via the medium to the optionally attached microphages on the inner walls of the bioreactors, and are likewise phagocytosed by the latter. This leads to the directed phenotypical changes in the myofibroblasts and an enhanced local remodeling activity.

Five days after starting myofibroblast colonization, endothelial cells are placed on the myofibroblasts which are already confluent and have penetrated into the depth by then, and cultivated for a further 2 days. The outer structures are then already replaced by an autologous matrix, and an internal core structure with a variably adjustable half-life is still retained.

The implant can be implanted even at this time. The transformation processes are completed in vivo. The macrophage-activating particles have already been removed by the phagocytic cells at this time.

1. The use of a natural tissue matrix which has undergone recipient-specific transformation for producing an individual vein valve prosthesis, wherein the natural tissue matrix has not been acellularized.

2. The use as claimed in claim 1, wherein the recipient-specific transformation comprises a colonization of the matrix by recipient-compatible cells.

3. The use as claimed in claim 1, wherein the recipient-specific transformation comprises colonization of the matrix by autologous cells of the prosthesis recipient.

4. The use as claimed in claim 1, wherein the matrix is a xenogeneic matrix.

5. The use as claimed in claim 1, wherein the matrix is an allogeneic matrix.

6. The use as claimed in claim 1, wherein the matrix is a natural vein valve.

7. The use as claimed in claim 1, wherein the matrix is a natural artery valve.

8. The use as claimed in claim 1, wherein the matrix contains microparticles.

9. The use as claimed in claim 1, wherein the matrix contains phagocytozable microparticles.

10. The use as claimed in claim 1, wherein the matrix contains degradable microparticles.

11. The use as claimed in claim 1, wherein the vein valve prosthesis is constructed from matrix material which has undergone recipient-specific transformation.

12. The use as claimed in claim 1, wherein the vein valve prosthesis comprises at least one valve leaflet.
